(19) United States
(12) Patent Application Publication
(10) Pub. No.: US 2009/0105812 A1
(43) Pub. Date: Apr. 23, 2009

(54) DEVICE AND METHOD FOR USE IN AORTIC VALVE DISEASE TREATMENT

(75) Inventors: Boris Levin, Rehovot (IL); Alexander Rejabek, Rehovot (IL); Alexander Lerner, Rehovot (IL); Sofia Levin, Rehovot (IL)

Correspondence Address: BROWDY AND NEIMARK, P.L.L.C.;624 NINTH STREET, NW, SUITE 300, WASHINGTON, DC, 20001-5303, US

(73) Assignees: Yeda Research & Development Ltd. The Weizmann Institute of Science, Rehovot (IL); Mor Research Applications Ltd., Rehovot (IL)

(21) Appl. No.: 12/255,558

(22) Filed: Oct. 21, 2008

Related U.S. Application Data
(63) Continuation of application No. 11/910,502.
(60) Provisional application No. 60/667,684, filed on Apr. 4, 2005.

(51) Int. Cl. A61F 2/24

(52) U.S. Cl. 623/2.2

(57) ABSTRACT

A device is presented for assisting an insufficient aortic valve. The device is configured as an auxiliary valve and comprises a resilient deformable sleeve-like valve, deformation of the sleeve-like valve shifting the valve between its closed and opened positions.

### FIELD OF THE INVENTION

This invention relates to medical devices intended for treating patients with aortic valve disease.

### BACKGROUND OF THE INVENTION

Aortic valve disease is one of the most widespread heart diseases in humans. This disease is caused by stretching the aortic valve in the course of stenosis treatment or natural development of insufficiency.

The aortic valve is located between the left ventricle and the ascending aorta, its main function being the prevention of the oxygen-enriched blood from returning to the heart during the diastolic relaxation phase while not hindering blood flow during the heart's constriction phase. In other words, the aortic valve is open during systole and closed during diastole. However, such a normal function may be disturbed as a result of various heart disorders, of which there are two major types: stenosis, whereas the aortic valve is incompletely open during systole, and aortic regurgitation where the aortic valve is incompletely closed during diastole. Regurgitation is due to incompetence of the aortic valve or any disturbance of the valvular apparatus (e.g., leaflets, annulus of the aorta) resulting in diastolic flow of blood into the left ventricular chamber.

Incompetent closure of the aortic valve can result from intrinsic disease of the cusp, diseases of the aorta, or trauma. Aortic regurgitation may be a chronic disease process, or it may occur acutely, resulting in heart failure. The most common cause of chronic aortic regurgitation used to be rheumatic heart disease, but presently it is most commonly bacterial endocarditis. In developed countries, it is caused by dilatation of the ascending aorta (e.g., aortic root disease, aortoannular ectasia).

Aortic valve insufficiency may also be caused by artificial stretching of the aortic valve during the course of stenosis treatment, using balloon valvuloplasty or a similar technique relying on percutaneous catheterization. Such artificially induced acute insufficiency, especially in elder patients, leads to a stabilization of the patient's condition but must be subsequently corrected.

Irrespective of its cause—either naturally developed or surgically induced—diastolic reflux through the aortic valve, can lead to left ventricular volume overload. The severity of aortic regurgitation is dependent on the diastolic valve area, the diastolic pressure gradient between the aorta and left ventricle, and the duration of diastole. An increase in systolic stroke volume and low diastolic aortic pressure produces an increased pulse pressure. In many cases, the severity of the disease warrants the use of a surgical procedure, particularly that of aortic valve replacement with an artificial valve. Various models of mechanical and bioprosthetic artificial aortic valves have been developed and introduced into clinical practice.

The number of aortic valve replacement procedures in 2002 amounted to almost 80,000 in the USA alone. However, such a procedure, being open-heart surgery, involves significant postoperative risks, is not suitable for all patients, and is rather costly.

### SUMMARY OF THE INVENTION

There is a need in the art for a less invasive and surgically simple alternative to the conventional technique of aortic valve replacement, by providing a novel method and device for assisting an insufficient aortic valve.

The technique of the present invention takes advantage of the fact that an incompetent aortic valve does not actually need to be removed, provided that its function of blocking the diastolic blood flow is performed with the assistance of an artificial device.

The present invention is intended for aiding patients with an incompetent aortic valve by means of providing an auxiliary valve capable of blocking the return blood flow from the aorta to the left ventricle during diastole. Both a device and a method of using thereof are presented. The technique of the present invention utilizes an auxiliary valve rather than a replacement valve. Such an auxiliary valve is introduced into patient's body using a customary catheterization technique, while a leaking natural aortic valve is left in place, thus eliminating the need for open-heart surgery.

The auxiliary valve is rendered, in the preferred embodiment of the invention, as a reverse (normally closed) sleeve type valve with a hyperboloidal surface, having an open proximal end and an open distal end, and manufactured of a biocompatible resilient memory material, for example a polymer or composite material such as polyurethanes, polyetherurethanes, polysulfones, PEEK polymers, etc. These may be shape-memory thermoplastic polymers. The valve (sleeve) has opposite open distal and proximal ends. The proximal end is intended to be affixed to a stent for insertion at a location proximal to the natural aortal valve and is preferably formed with a rigid ring (termed “proximal ring”). The opposite, distal end of the sleeve is preferably also made more rigid than the sleeve body, which can be achieved by using a distal ring or by making the sleeve material thicker along the circumference of the distal end. The sleeve is preferably pre-twisted (e.g., in a clockwise direction), so that its hyperboloidal surface is degraded into a double near-conical surface with a very thin neck that effectively prevents any liquid flow therethrough.

When pressure equilibrium exists at both ends of the auxiliary valve, or a pressure gradient is directed from the aorta towards the natural aortic valve or the left ventricle (i.e. form the distal end to the proximal end of the sleeve), it causes a further clockwise twisting of the sleeve and even tighter closure of the sleeve neck during diastole. On the other hand, when a pressure gradient is directed from the left ventricle through the natural aortic valve towards the aorta (i.e. from the proximal end towards the distal end of the auxiliary valve sleeve), the auxiliary valve is untwisted (e.g. in a counter-clockwise direction), and its hyperboloidal surface develops into a nearly cylindrical one, thus allowing for an unhindered blood flow during systole.

Thus, the proximal end of the sleeve (located at the natural aorta valve or the left ventricle side) is supported, and cannot move. Between the sistola and diastola stages of the cycle, no pressure gradient exists. The “natural” (memorized) twisted form of the sleeve-like auxiliary valve is preserved. At the diastola stage of the cycle, pressure equilibrium exists inside and outside the sleeve at proximal parts of the valve. The distal end of the valve (located inside the aorta) is under pressure from the outside of the sleeve. This force is directed so that the sleeve is twisted closing the neck. On the other hand, at the sistola stage, when a pressure gradient is directed from the left ventricle through the natural aortic valve towards the aorta (i.e. from the proximal end towards the distal end of the auxiliary valve sleeve), there is a pressure at the proximal end of the valve from the inside of the sleeve. This force is directed so that the sleeve is twisted, opening the neck.

Applying the technique of the present invention provides for eliminating a diastolic reflux through the insufficient aortic valve without replacing such a valve.

Thus, the present invention provides for a device for assisting an insufficient aortic valve, the device comprising a resilient deformable sleeve-like valve, such that the deformation of the valve caused by a blood flow pressure shifting the valve between its closed and opened positions.

When the device is put in operation (inserted into the patient's body), the proximal ring is placed stationary inside the aorta just above the aorta root, while the distal end (ring) is located further in the ascending part of the aorta and is unconstrained with regard to its longitudinal and rotational movement other than due to its attachment to the sleeve.

Preferably, the diameter of the sleeve-like valve at the distal end thereof (e.g. diameter of the distal ring) is smaller than the diameter of the proximal ring, so that the surface of the pliable sleeve is slightly conical rather than cylindrical.

The distal ring is initially, prior to using the device, rotated with regard to the stationary proximal ring, so that the resilient sleeve is deformed while progressively acquiring geometrical forms from that of an axis-cut cone through that of a hyperboloid of the revolution to eventually becoming that of two cones with mutually adjacent tips, thus forming a tight neck at some point along the sleeve's longitudinal axis.

The present invention also provides a method of using the above device which functions as a normally closed auxiliary valve operating in line with an incompetent natural aortic valve, so that it prevents diastolic reflux through the incompetent natural aortic valve while not obstructing the systolic blood flow.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 schematically shows a device of the present invention being positioned inside the aorta with regard to other parts of the heart, which are designated by their customary abbreviations, whereas a sleeve valve is closed during diastola;

FIG. 2 schematically shows the device of the present invention being positioned inside the aorta with regard to other parts of the heart, whereas the sleeve valve is opened during systole;

FIG. 3 schematically shows the principles of operation of the sleeve valve employed in the invention and illustrates its subsequent states from “closed” to “opened” and back to “closed”, where curved arrows show the direction of a distal ring rotational movement, while vertical arrows show the direction of the distal ring longitudinal movement.

### DETAILED DESCRIPTION OF THE INVENTION

The present invention provides a device and method intended for aiding patients with an incompetent aortic valve by means of providing an auxiliary valve capable of blocking the return blood flow (reflux) from the aorta to the left ventricle during diastole. The technique of the present invention is based on employing an auxiliary aortic valve operating in line with an intact incompetent natural aortic valve, instead of employing an artificial replacement valve.

Such an auxiliary aortic valve is configured to facilitate its introduction into the patient's body and fixation of the device at its intended place just above the aorta root without any open-heart surgery but rather using a well-established, much less traumatic and more affordable catheterization technique. Such an approach is made possible owing to the abovementioned fact of the incompetent (leaking) natural aortic valve being left intact rather than being removed surgically.

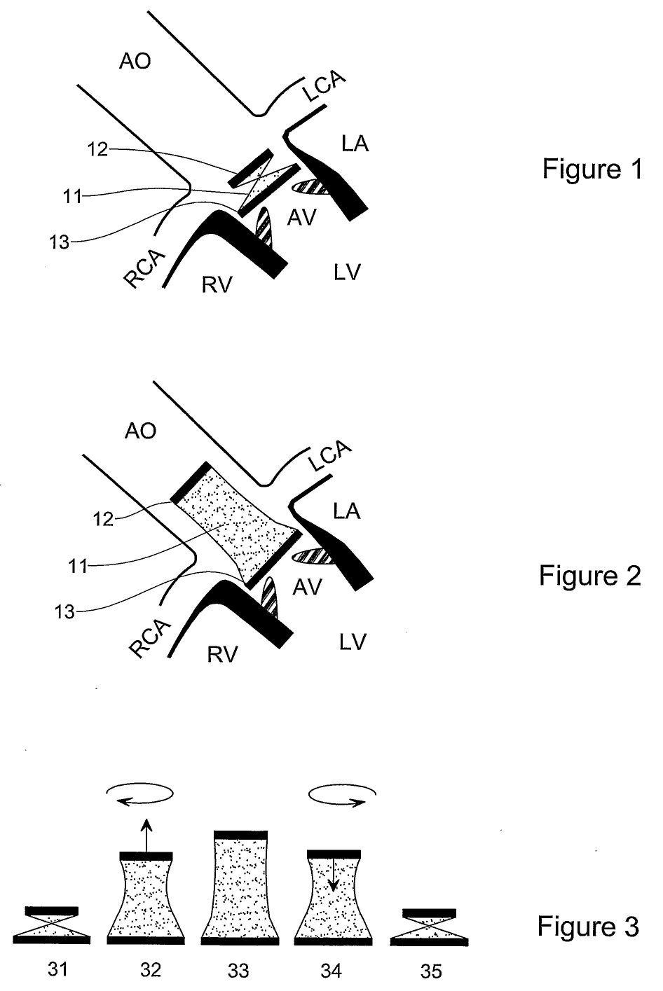

Referring to FIGS. 1 and 2, there is schematically illustrated a device of the present invention for assisting an insufficient aortic valve. The device includes an auxiliary aortic valve 11 configured according to the present invention as a resilient deformable sleeve-like valve such that a deformation of the valve 11 shifts it between its closed and opened positions. The valve 11, is configured as a reverse (normally closed) sleeve type valve, which in the present example has a hyperboloidal surface, and has an open proximal end and an open distal end.

FIG. 1 illustrates the valve operational position during diastola (the sleeve valve is closed), and FIG. 2 shows the operational position during systole (the sleeve valve is open). As indicated above, the aortic valve is made of a resilient material.

At its opposite ends, the sleeve 11 is attached to a proximal ring 13 and a distal ring 12, respectively. The proximal ring 13 is placed stationary inside the aorta just above the aorta root, while the distal ring 12 is located further in the ascending part of the aorta. The only constraint for longitudinal and rotational movement of the distal ring 12 is due to its attachment to the sleeve 11. Otherwise the distal ring 12 would turn around its central axis when a rotational moment is applied by the sleeve 11. In this case, the distal ring 12 also travels longitudinally following either elongation or contraction of the sleeve 11.

Turning now to FIG. 3, the operational principles of the sleeve valve 11 are schematically illustrated, showing the subsequent states of the sleeve from “closed” to “opened” and back to “closed”. Here, curved arrows correspond to the direction of the rotational movement of the distal ring 12, and vertical arrows correspond to the direction of the longitudinal movement of the distal ring 12.

The sleeve 11 is deformable by twisting. In the course of such deformation (e.g., in a counter-clockwise direction), the sleeve 11 progressively acquires different geometrical forms, starting from the form of an axis-cut cone. Then, the sleeve 11 passes through various stages of a hyperboloid of the revolution, and eventually takes the form of two cones with mutually adjacent tips, shown in the figure as position 31. In the course of this movement, the sleeve further changes its geometrical form from that of two cones with mutually adjacent tips through various stages of a hyperboloid of the revolution (position 32), eventually acquiring the form of an axis-cut cone (position 33). The resiliency of the sleeve causes it to start deforming (twisting) in a counter-clockwise direction (position 34), eventually forming the closed state of the valve (position 35).

Prior to inserting the device into the patient's body (e.g., at the device manufacturing stage), the distal ring 12 is rotated with respect to the proximal ring 13, so as to cause the deformation (twisting) of the sleeve 11, as described above. When the sleeve 11 takes the form of two cones with mutually adjacent tips (position 31), a tight neck is formed at a certain location along the sleeve's longitudinal axis where the two cones meet with their respective tips. This tight neck effectively prevents any liquid flow therethrough. This state of the device exists during diastole (FIG. 1). In this state, either a pressure equilibrium exists at both ends of the auxiliary valve 11, or a pressure gradient is directed from the aorta towards the natural aortic valve. In any case, it causes a further rotational deformation (twisting) of the sleeve 11 and therefore even tighter closure of the sleeve neck during diastole, any possible reflux through an incompletely closed natural aortic valve notwithstanding.

The above-described closed state of the valve exists until a pressure gradient becomes directed from the left ventricle towards the aorta, which takes place during systole when the natural aortic valve is open. The force created by such pressure gradient is applied to the inner surface of the sleeve and causes its rotational movement in the direction opposite to the direction in which the sleeve was deformed (twisted), as described above, and the sleeve changes its geometrical form from that of two cones with mutually adjacent tips through various stages of a hyperboloid of the revolution (position 32), eventually acquiring the form of the axis-cut cone (position 33). This latter state of the device is shown in FIG. 2.

It is apparent from FIG. 2 that in such a state of the device the auxiliary valve is fully open, allowing for an unhindered blood flow from the left ventricle to the aorta during systole. Owing to a conical, rather than cylindrical, shape of the resilient sleeve 11, the systolic blood flow to the coronary arteries encounters no obstruction. At the end of systole, the aorta-directed pressure gradient ceases to exist, and the resiliency of the sleeve 11 causes it to start deforming (twisting) in a counter-clockwise direction (position 34), eventually forming the closed state of the valve (position 35).

The above-described process is repeated cyclically, following the systole-diastole cycle of the heart, and does not rely on any maintenance or energy supply. If however adjustment or removal of the device is needed, it may be achieved by relatively easy means using the same catheterization technique as for the device insertion.

1. A device for assisting an insufficient aortic valve, the device comprising a resilient deformable sleeve-like valve, deformation of the valve shifting the valve between its closed and opened positions.

2. The device of claim 1, wherein the sleeve-like valve is made of a shape-memory material.

3. The device of claim 1, wherein the sleeve has opposite open proximal and distal ends, which, when the device is in operation, are associated with, respectively, an aorta part just above the aorta root, and an ascending part of the aorta, with the proximal end being fixed and the distal end being freely located.

4. The device of claim 3, wherein the sleeve-like valve is pre-twisted to have a normally closed position effectively preventing any liquid flow therethrough, and be shiftable into the opened position by a pressure gradient in a direction from the proximal end to the distal end.

5. The device of claim 4, wherein the sleeve-like valve is configured such that a state of pressure equilibrium at the proximal and distal ends or a state of a pressure gradient directed from the distal end towards the proximal end results in further twisting of the sleeve-like valve material towards its closed position to provide even tighter closure during diastole, and a state of the pressure gradient directed from the proximal end to the distal end results in untwisting of the sleeve-like valve material to provide its open position allowing for an unhindered blood flow during systole.

6. The device of claim 3, comprising a proximal ring and a distal ring attached to the proximal and distal ends of the sleeve, respectively.

7. The device of claim 6, wherein, when in the operation of the device, the proximal ring is placed stationary inside an aorta just above the aorta root, and the distal ring is located further in an ascending part of the aorta and is unconstrained with regard to its longitudinal and rotational movement other than due to its attachment to the sleeve.

8. The device of claim 2, wherein the sleeve-like valve is pre-twisted to have a normally closed position effectively preventing any liquid flow therethrough, and be shiftable into the opened position by a pressure gradient in a direction from the proximal end to the distal end.

9. The device of claim 8, wherein the sleeve-like valve is configured such that a state of pressure equilibrium at the proximal and distal ends or a state of a pressure gradient directed from the distal end towards the proximal end results in further twisting of the sleeve-like valve material towards its closed position to provide even tighter closure during diastole, and a state of the pressure gradient directed from the proximal end to the distal end results in untwisting of the sleeve-like valve material to provide its open position allowing for an unhindered blood flow during systole.

10. The device of claim 3, wherein the diameter of the distal end is smaller than the diameter of the proximal end, the surface of the pliable sleeve being thereby slightly conical rather than cylindrical.

11. The device of claim 3, wherein the sleeve is put into its initial operational position, prior to putting the device in operation, by rotating the distal end with respect to the stationary proximal end, thereby causing the deformation of the resilient sleeve to progressively take geometrical forms starting from an axis-cut cone through a hyperboloid of the revolution to eventually becoming that of two cones with mutually adjacent tips, thus forming a tight neck at some point along a longitudinal axis of the sleeve.

12. The device of claim 1, configured and operable as a normally closed auxiliary valve for operating in line with an incompetent natural aortic valve, thereby preventing diastolic reflux through the incompetent natural aortic valve while not obstructing systolic blood flow.

13. A method for operating a device for assisting an insufficient aortic valve, the method comprising accommodating the device of claim 1 inside the patient's body such that a proximal ring attached to a proximal end of the resilient sleeve is placed stationary inside an aorta just above the aorta root, and a distal ring attached to a distal end of said sleeve is located further in an ascending part of the aorta and is unconstrained with regard to its longitudinal and rotational movement other than due to its attachment to the sleeve, thereby providing a normally closed auxiliary valve operating in line with an incompetent natural aortic valve, so as to prevent diastolic reflux through the incompetent natural aortic valve while not obstructing the systolic blood flow.
